(19) United States
(12) Patent Application Publication
(10) Pub. No.: US 2021/0093370 A1
(43) Pub. Date: Apr. 1, 2021

(54) FOOTSWITCH FOR AN ELECTROSURGICAL INSTRUMENT

(71) Applicant: GYRUS MEDICAL LIMITED, Cardiff (GB)

(72) Inventor: Richard HOODLESS, Cardiff (GB)

(73) Assignee: GYRUS MEDICAL LIMITED, Cardiff (GB)

(21) Appl. No.: 17/000,693

(22) Filed: Aug. 24, 2020

(30) Foreign Application Priority Data
Sep. 27, 2019 (GB) 1913989.8

(51) Int. Cl. A61B 18/12

(52) U.S. Cl. CPC A61B 18/1206; A61B 2018/00309; A61B 2018/124

(57) ABSTRACT

An improved footswitch is provided to control multiple functions of an electrosurgical instrument simultaneously. The footswitch comprises one or more pedals that are configured to control the functions of the instrument, wherein at least two of the functions can be activated at the same time. For example, the pedals may be arranged to activate a mechanical shaver function, and at the same time activate one or both of an RF cutting function and an RF coagulation function.

### TECHNICAL FIELD

Embodiments of the present invention described herein relate to a footswitch, and in particular to a footswitch for controlling the functions of an electrosurgical device.

### BACKGROUND TO THE INVENTION AND PRIOR ART

Surgical instruments, including radio frequency (RF) shaver electrosurgical instruments, have become well accepted in surgical procedures where access to the surgical site is restricted to a narrow passage, for example, in minimally invasive “keyhole” surgeries.

Combination RF shaver electrosurgical instruments comprise an electrode and shaver component, allowing one instrument to be used for both cutting and coagulation. Generic footswitches are known in the medical industry for controlling medical devices in operating rooms. For example, U.S. Pat. No. 8,143,540 discloses a multifunctional foot switch. The foot switch has two or more actuation elements, arranged such that the operator of the foot switch may select various devices functionally connected to the foot switch merely be repositioning the foot. U.S. Pat. No. 2,944,120 discloses an electric motor footswitch, and describes basic workings of a footswitch. U.S. Pat. No. 6,051,797 discloses a foot pedal switch for manually operating a medical diagnostic apparatus having a switch housing and a switching rocker with a neutral position that can be rotated around a rotational axle, with the rotational axle projecting from the housing such that it forms a bearing point of the foot pedal switch with its free end.

However, in a combination RF shaver device, a user may wish to combine an RF cut function and a mechanical shave function for an increased tissue removal effect, or an RF coagulation function and a shave function for haemostasis whilst mechanically debriding tissue. In the case of a footswitch control, a set-up needs to be provided to facilitate effective use to minimise effort and reduce the risk of human error.

### SUMMARY OF THE INVENTION

Embodiments of the present invention provide an improved set-up for a footswitch to control multiple functions of an electrosurgical instrument simultaneously. The footswitch comprises one or more pedals that are configured to control the functions of the instrument, wherein at least two of the functions can be activated at the same time. For example, the pedals may be arranged to activate a mechanical shaver function, and at the same time activate one or both of an RF cutting function and an RF coagulation function.

A first aspect of the present invention provides an electrosurgical system comprising an electrosurgical generator, wherein the generator comprises:
(i) a source of radio frequency (RF) power;
(ii) two or more electrical connections for connecting the generator to an electrosurgical instrument; and
(iii) a controller operable to cause the generator to supply power to the two or more connections; and
a footswitch comprising one or more pedals operable to send signals to the controller to activate the source of RF power, wherein the one or more pedals are operable to send a first signal to the controller to supply a direct current to a first connection, and at least a second signal to the controller to supply an RF electrosurgical signal to a second connection, and wherein the one or more pedals are configured to allow simultaneous activation of the direct current and the RF electrosurgical signal.

As such, the footswitch acts to control the supply of power by the generator to an electrosurgical instrument, the footswitch being operated so as to activate the supply of a direct current and an RF electrosurgical signal to the instrument. In use, this enables the user to activate both a mechanical function and an RF electrosurgical function of the instrument at the same time. For example, the user may wish to operate a mechanical shaving arrangement for cutting tissue, and at the same time operate an electrode assembly to provide a further tissue treatment such as cutting and/or coagulating. The footswitch is thus configured to allow both functions to be activated at the same time, through the actuation of one or more pedals.

The footswitch may comprise a first pedal, the first pedal being operable to send the first and second signals to the controller. That is to say, the first pedal is configured to activate both functions of the electrosurgical instrument.

The first pedal may be moveable from a first position to a second position to thereby send the first signal to the controller, and further moveable from the second position to a third position to subsequently send the second signal to the controller, such that the direct current and the RF electrosurgical signal are simultaneously supplied to the first and second connections respectively. As such, the first pedal can be depressed in two-stages to activate the two output signals. This allows the user to first activate the direct current, and then additionally activate the RF signal when required.

To facilitate the two-stage activation, the footswitch may comprise a first switch in communication with the controller, wherein movement of the first pedal to the second position places the first switch in a first state to thereby send the first signal to the controller, and wherein movement of the first pedal to the third position places the first switch in a second state to thereby send the second signal to the controller.

Alternatively, the footswitch may comprise a first switch and second switch in communication with the controller, wherein movement of the first pedal to the second position causes the first switch to send the first signal to the controller, and wherein movement of the first pedal to the third position causes the second switch to send the second signal to the controller

In another arrangement, the footswitch may comprise a first pedal, the first pedal being operable to send the first signal to the controller, and at least a second pedal, the second pedal being operable to send the second signal to the controller. As such, the footswitch comprises separate pedals for activating the direct current and RF signal. In some arrangements, the footswitch may comprise a third pedal configured to control the supply of a further RF signal to the second connection, such that the second and third pedals control different RF electrosurgical functions. For example, the second pedal may control the RF cutting function, and the third pedal may control the RF coagulating function. Alternatively, the second pedal may control both RF functions, wherein the selection of the RF waveform is controlled by some other means on the generator.

The first pedal may be configured to move between a first and second position to thereby send the first signal, and the second pedal may be configured to move between a first and second position to thereby send the second signal.

In some arrangements, the first pedal may be configured to move the second pedal to its second position when a user of the footswitch moves the first pedal to its respective second position. That is to say, the first pedal may comprise some internal or external actuation means that acts to move the second pedal with the first pedal, thereby activating the direct current and the RF electrosurgical signal at the same time.

In some arrangements, first pedal may be operable to send a third signal to the controller to stop supplying the direct current and the RF electrosurgical signal to the respective first and second connections. For example, in any of the above described arrangements, the release of the first pedal to its first position may deactivate both the direct current and the RF signal.

A position of the first pedal may be detectable to a user by receiving feedback by at least one of the following:
i. a tactile footswitch “click”;
ii. an audible footswitch “click”;
iii. an audible generator tone change;
iv. a visual icon change on a general user interface or a display;
v. a change in force to move the first pedal.

This is particularly important for arrangements in which the first pedal comprises a two-stage activation so that the user knows which position the pedal is in, and thus which of the output signals is activated.

The first connection may be configured to connect the generator to drive componentry of a shaver arrangement, such that supply of the direct current to the first connection causes the drive componentry to drive the shaver arrangement.

The second connection may be configured to connect the generator to an electrode assembly.

The RF electrosurgical signal may comprise an RF cutting waveform. The RF electrosurgical signal may comprise an RF coagulation waveform. Alternatively, the RF electrosurgical signal may be a blended RF signal comprising an RF cutting waveform and an RF coagulation waveform.

In some arrangements, the system further comprises an electrosurgical instrument, the electrosurgical instrument comprising an end-effector having a shaver arrangement and an electrode assembly.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 is a schematic diagram of an electrosurgical instrument according to an embodiment of the present invention;

FIG. 2 is a plan view of a footswitch according to an embodiment of the present invention;

FIGS. 3A-C are cross-sectional side views of a footswitch according to an embodiment of the present invention;

FIGS. 4A-C are cross-sectional side views of a footswitch according to an embodiment of the present invention;

FIGS. 5A-C are cross-sectional side views of a footswitch according to an embodiment of the present invention.

### DESCRIPTION OF THE EMBODIMENTS

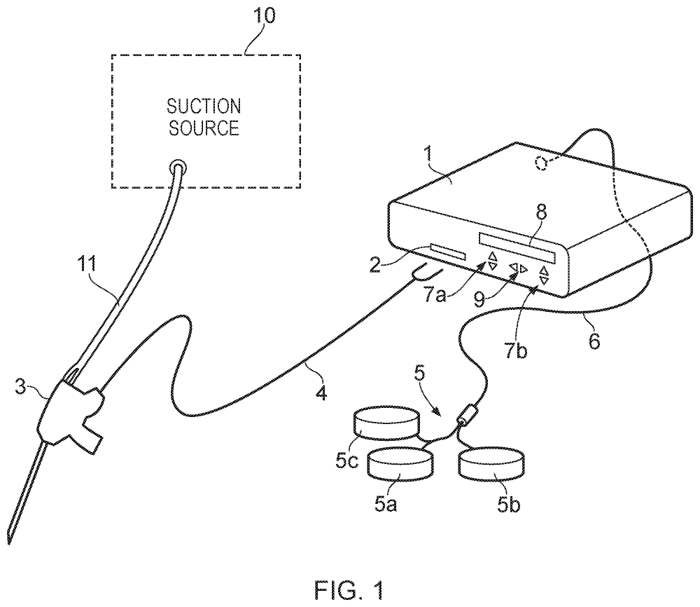

Referring to the drawings, FIG. 1 shows electrosurgical apparatus including an electrosurgical generator 1 having an output socket 2 providing a radio frequency (RF) output, via a connection cord 4, for an electrosurgical instrument 3 having an end effector configured to provide a mechanical shaving function, as well as electrosurgical cutting and coagulation functions. The instrument 3 may also comprise irrigation and suction tubes 11 which are connected to an irrigation fluid and suction source 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has three footswitches 5a, 5b and 5c for selecting a mechanical shaving mode, coagulation mode or a cutting mode of the generator 1 respectively. The generator front panel has push buttons 7a and 7b for respectively setting cutting or coagulation power levels, which are indicated in a display 8. Push buttons 9 are provided as an alternative means for selection between the different modes, as will be described in more detail below.

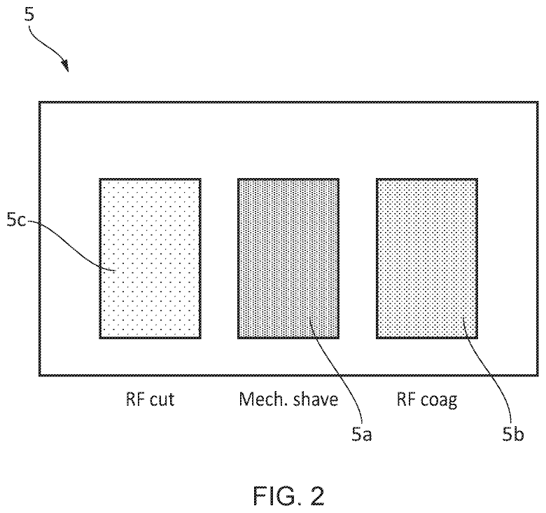

FIG. 2 shows the footswitch unit 5 in more detail. Each pedal 5a, 5b, 5c can be depressed individually to activate its assigned function in isolation by sending a signal to the generator 1 to activate the mechanical shaving function of the end effector, for example, by supplying a direct current to drive componentry for a shaver, or to start sending the relevant RF signal(s) to activate the electrosurgical functions of the end effector. For example, pedal 5a may control the mechanical shaving function, pedal 5b may control the coagulation function and pedal 5c may control the cutting function. In this respect, the generator 1 also comprises control circuitry (software plus switching relays) constituting selection means for changing which of the output connections become “live”, and then controlling the on/off supply of the RF signals to the live output connections.

A user may wish to simultaneously combine two or more functions. For example, a user may wish to combine the shaving and cutting functions together for an increased tissue removal effect. Alternatively, a user may wish to combine the shaving and coagulating functions for haemostasis whilst mechanically debriding tissue. In some situations, the user may wish to combine the mechanical shave, and a blend of RF cutting and coagulation signals.

In a first mode, to activate two or more functions at once, a user depresses two or more pedals 5a, 5b, 5c simultaneously. This sends a signal to the generator 1 to activate the mechanical shaving function, and start sending an RF cutting and/or coagulation signal to the instrument 3. To stop the two or more functions, the user releases the two or more pedals 5a, 5b, 5c. For example, if the user wants to activate the shave and cutting functions, the user will depress pedals 5a and 5c, and then release those pedals when finished.

In a second mode, to activate two functions at once, a user depresses and releases the first pedal e.g. 5a controlling the first shaving function, then depresses the second pedal e.g. 5b controlling the second coagulation function. To stop the two functions, the user releases the second pedal 5b (stopping the second function), and then depresses and releases the first pedal 5a (stopping the first function). Alternatively, both the first and second functions may be stopped when the second pedal 5b is released. That is to say, when the user releases pedals 5b or 5c, corresponding to the RF coagulating and cutting functions respectively, the mechanical shaving function may stop automatically at the same time.

The footswitch unit 5 may also be configured such that a user may select either the first or the second mode described above. This could be either by an additional pedal (not shown) on the footswitch 5, or on the control console of the generator 1, for example, via push buttons 9.

Alternatively, to activate two or more functions at once, pedal 5a may be configured to be depressed to a first position and a second position. In the first position, the first function, for example, the shaving function, is activated, and in the second position one or both of the second and third function, for example, the cutting and/or coagulating RF, are additionally activated. To facilitate this arrangement, the initial starting position of pedal 5a will be higher than the starting positions of the second and third pedals 5b, 5c.

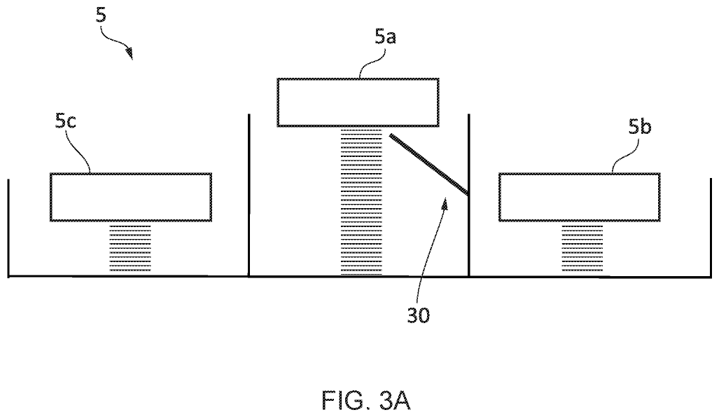

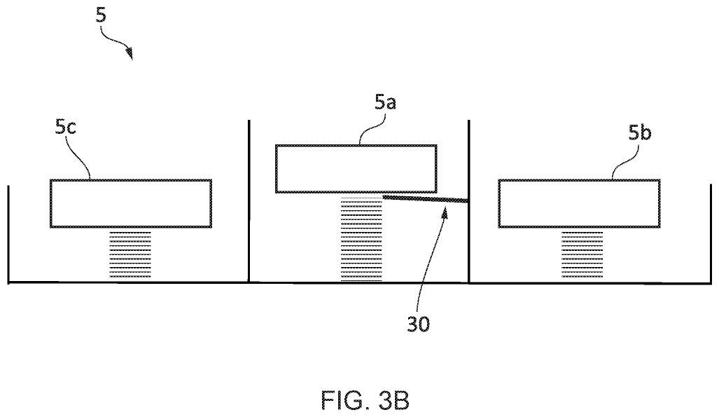

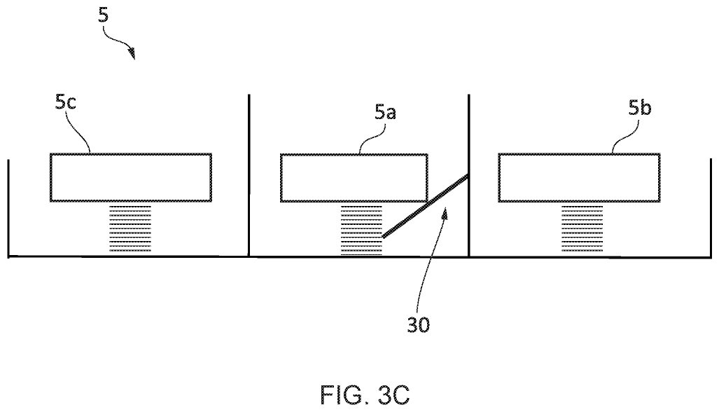

This two stage activation may be carried out in a number of different ways. In a first example, as illustrated by way of example in FIGS. 3A-C, the first pedal 5a may be provided with an internal three position switch 30 in the first pedal 5a, the switch having two “on” states such that the first “on” state activates the first function and the second “on” state additionally activates one or both of the second and third function. As shown in FIG. 3A, pedal 5a is in its starting position and the switch 30 is in the “off” state, such that none of the output connections to the instrument 3 are “live”. In FIG. 3B, the pedal 5a is moved to its first position such that the switch 30 is moved to its first “on” state, which sends a signal to the generator 1 to activate the mechanical shaving function. In FIG. 3C, the pedal 5a is moved to its second positon such that the switch is moved to its second “on” state, which sends a signal to the generator 1 to start sending one or both of the RF cutting and coagulating signals to the instrument 3.

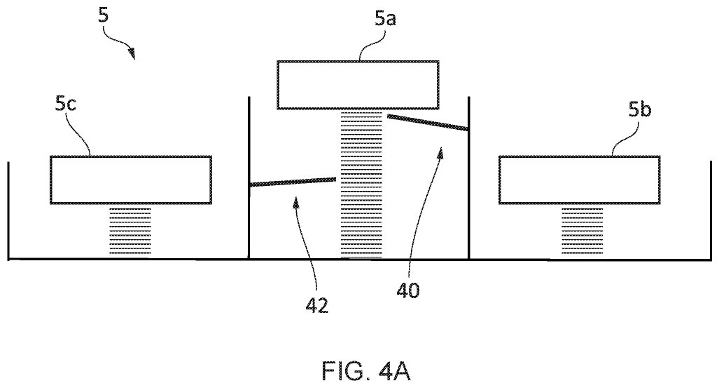

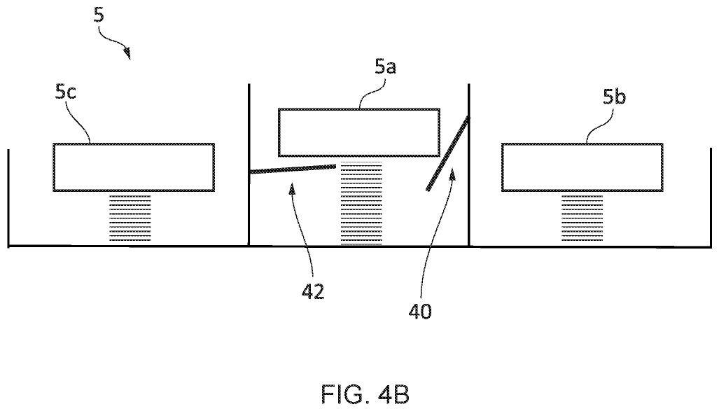

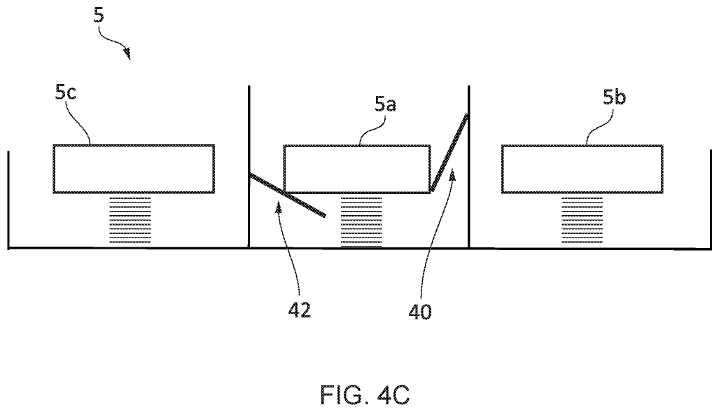

In another arrangement, the two-stage activation of the first pedal 5a may be implemented by providing at least two internal switches 40, 42 within the first pedal 5a, as illustrated by way of example in FIGS. 4A-C. As shown in FIG. 4A, pedal 5a is in its starting position and the both switches 40, 42 are in the “off” state, such that none of the output connections to the instrument 3 are “live”. In FIG. 4B, the pedal 5a is moved to its first position such that the first switch 40 is moved to its first “on” state, whilst the second switch 42 remains in its “off” state, which sends a signal to the generator 1 to activate the mechanical shaving function. In FIG. 4C, the pedal 5a is moved to its second positon such that the second switch 42 is also moved to its “on” state, which sends a signal to the generator 1 to start sending one or both of the RF cutting and coagulating signals to the instrument 3.

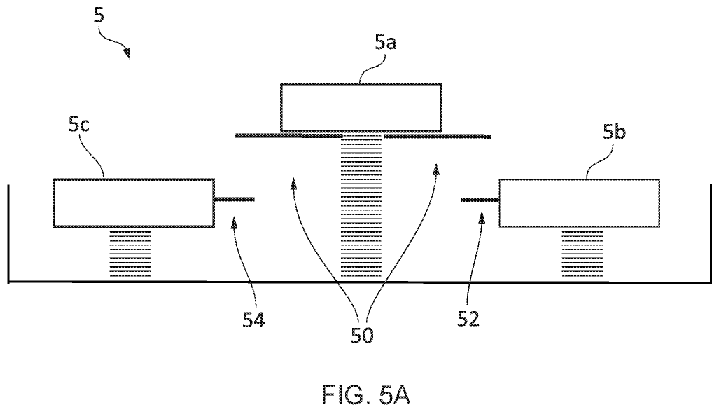

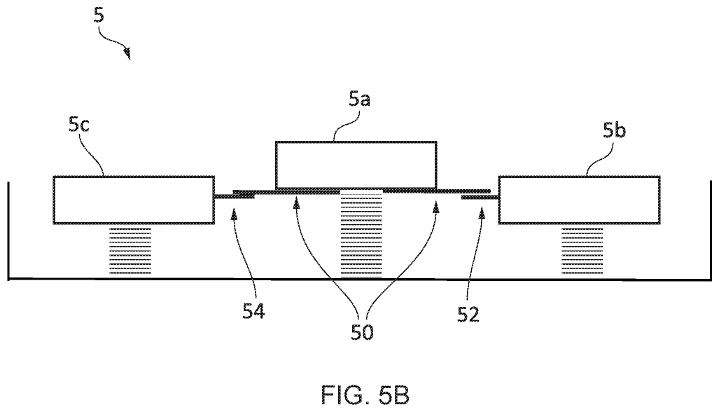

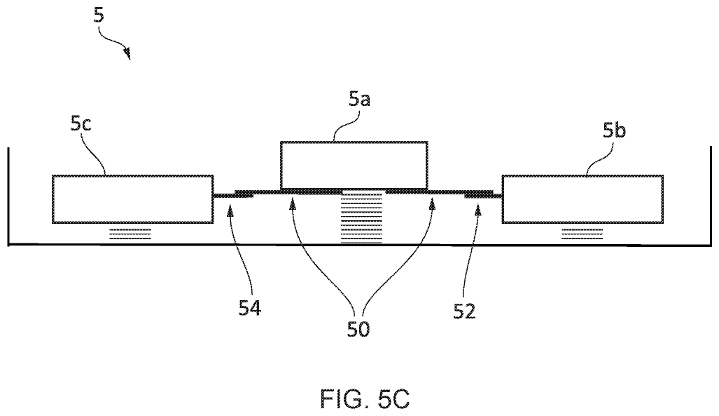

In yet a further arrangement, the two-stage activation may be implemented an internal or external structural feature on the first pedal 5a which, when the first pedal 5a is depressed to the second position, automatically mechanically depresses one or both of the second 5b and third 5c pedals in order to additionally activate one or both of the second and third functions respectively. An example of this arrangement is provided by FIGS. 5A-C, wherein each of the second and third pedals 5b, 5c are provided with a projection 52, 54 extending from a side wall in the direction of the first pedal 5a. The first pedal 5a is provided with one or more arms 50 extending from its base in the direction of the second and/or third pedal 5b, 5c. As shown FIG. 5A, the first pedal 5a is in its starting position such that neither the shaving nor the cutting/coagulation functions have been activated. In FIG. 5B, the pedal 5a is moved to its first position such that some switching means (not shown) has been triggered, thereby sending a signal to the generator 1 to activate the mechanical shaving function. In FIG. 5C, the pedal 5a is moved to its second positon, such that the arms 50 have cooperated with the projections 52, 54 to thereby mechanically depress the second and/or third pedals 5b, 5c to thereby connect electrical connections A and C through some further switching mean (not shown) and thus sending a signal to the generator 1 to start sending one or both of the RF cutting and coagulating signals to the instrument 3. As discussed previously, the user may be able to select one or both of the RF cutting and coagulating functions through an additional foot peddle or a control on the control console of the generator 1. That is to say, the user can control the generator 1 to send one or both of the RF cutting and coagulation signals to the live output connections, such that when the pedal 5a is in its second position, either the RF cutting signal is sent to the instrument 3, the RF coagulation signal is sent to the instrument 3, or a blend of the RF cutting and coagulation signals are sent to the instrument 3.

As part of the two-stage activation, the position of the first pedal 5a may be detectable to a user by receiving feedback through any suitable means. For example, the first pedal 5a may be provided with a tactile or audible footswitch “click”, which may be provided by one of the switching mechanisms described above, or a separate click switch mechanism. Alternatively, an audible generator tone may change when the activated function switches from just the first shaving function to the first shaving function and one or both of the second and third functions. A visual icon change on a general user interface or a display, for example, of the control console on the generator 1 may be provided. Alternatively, a change in the force required to depress the first pedal 5a to the second position compared with that required to depress the first pedal 5a to the first position. This change in force may be caused by an additional internal spring, or in response to one of the switching mechanisms described above being compressed in the first pedal 5a. For example, in the arrangement shown in FIGS. 5A-C, the depression of the adjacent second 5b and third 5c pedals, which contain their own switches and/or internal springs, may result in an increase in force required to move the first pedal 5a from the first position to the second position.

The system may be further configured to prevent accidental activation of an adjacent pedal by locking either the RF coagulating and cutting functions, or the shaving function. This could either be by an additional pedal (not shown) on the footswitch, or on the control console of the generator 1.

Various modifications whether by way of addition, deletion, or substitution of features may be made to above described embodiment to provide further embodiments, any and all of which are intended to be encompassed by the appended claims.

In the examples described herein, the footswitch comprises three pedals, however, it will be appreciated that the footswitch may comprise any suitable number of pedals. For example, the footswitch may comprise two foot pedals, one for activating the mechanical shaving function and one for activating one or both of the electrosurgical cutting and coagulation functions. As described above, selection of one or both of the electrosurgical cutting and coagulation functions may be facilitated by a further pedal on the footswitch, or on the console of the generator 1.

1. An electrosurgical system comprising:
an electrosurgical generator, wherein the generator comprises:
(i) a source of radio frequency (RF) power;
(ii) two or more electrical connections for connecting the generator to an electrosurgical instrument; and
(iii) a controller operable to cause the generator to supply power to the two or more connections; and
a footswitch comprising one or more pedals operable to send signals to the controller to activate the source of RF power, wherein the one or more pedals are operable to send a first signal to the controller to supply a direct current to a first connection, and at least a second signal to the controller to supply an RF electrosurgical signal to a second connection, and wherein the one or more pedals are configured to allow simultaneous activation of the direct current and the RF electrosurgical signal.

2. An electrosurgical system according to claim 1, wherein the footswitch comprises a first pedal, the first pedal being operable to send the first and second signals to the controller.

3. An electrosurgical system according to claim 2, wherein the first pedal is moveable from a first position to a second position to thereby send the first signal to the controller, and further moveable from the second position to a third position to subsequently send the second signal to the controller, such that the direct current and the RF electrosurgical signal are simultaneously supplied to the first and second connections respectively.

4. An electrosurgical system according to claim 3, wherein the footswitch comprises a first switch in communication with the controller, wherein movement of the first pedal to the second position places the first switch in a first state to thereby send the first signal to the controller, and wherein movement of the first pedal to the third position places the first switch in a second state to thereby send the second signal to the controller.

5. An electrosurgical system according to claim 3, wherein the footswitch comprises a first switch and second switch in communication with the controller, wherein movement of the first pedal to the second position causes the first switch to send the first signal to the controller, and wherein movement of the first pedal to the third position causes the second switch to send the second signal to the controller

6. An electrosurgical system according to claim 1, wherein the footswitch comprises:
a first pedal, the first pedal being operable to send the first signal to the controller; and
at least a second pedal, the second pedal being operable to send the second signal to the controller.

7. An electrosurgical system according to claim 6, wherein the first pedal is configured to move between a first and second position to thereby send the first signal, and wherein the second pedal is configured to move between a first and second position to thereby send the second signal.

8. An electrosurgical system according to claim 7, wherein the first pedal is configured to move the second pedal to its second position when a user of the footswitch moves the first pedal to its respective second position.

9. An electrosurgical system according to claim 2, wherein the first pedal is operable to send a third signal to the controller to stop supplying the direct current and the RF electrosurgical signal to the respective first and second connections.

10. An electrosurgical system according to claim 2, wherein a position of the first pedal is detectable to a user by receiving feedback by at least one of the following:
i. a tactile footswitch “click”;
ii. an audible footswitch “click”;
iii. an audible generator tone change;
iv. a visual icon change on a general user interface or a display;
v. a change in force to move the first pedal.

11. An electrosurgical system according to claim 1, wherein the first connection connects the generator to drive componentry of a shaver arrangement, such that supply of the direct current to the first connection causes the drive componentry to drive the shaver arrangement.

12. An electrosurgical system according to claim 1, wherein the second connection connects the generator to an electrode assembly.

13. An electrosurgical system according to claim 1, wherein the RF electrosurgical signal comprises an RF cutting waveform.

14. An electrosurgical system according to claim 1, wherein the RF electrosurgical signal comprises an RF coagulation waveform.

15. An electrosurgical system according to claim 1, wherein the RF electrosurgical signal is a blended RF signal comprising an RF cutting waveform and an RF coagulation waveform.

16. An electrosurgical system according to claim 1, further comprising an electrosurgical instrument, the electrosurgical instrument comprising an end-effector having a shaver arrangement and an electrode assembly.
